SEQUENCE LISTING (16 sequences)

SEQ ID NO: 1 - DNA, 1463 bp, Mus musculus
  CDS (280)..(1134)
aaaattgaaacaagtgcaggtgttcgcgggcacctaagcctccttcttaaggcagtcctccaggccaatgatggctccagggtaaaccacgtggggtgccccagagcctatggcacggcggccggcttgtccccagccagcctctggttccccaggagagcagtggagaactgtcaaagcgatctggggtggcgtagagagtccgcgagccacccagcgcctaaggcctggcttgtagctccgacccggggctgctggccccaagtgccggctgccaccatgaacagtgaggagcagtactacgcggccacacagctctacaaggacccgtgcgcattccagaggggcccggtgccagagttcagcgctaacccccctgcgtgcctgtacatgggccgccagcccccacctccgccgccaccccagtttacaagctcgctgggatcactggagcagggaagtcctccggacatctccccatacgaagtgcccccgctcgcctccgacgacccggctggcgctcacctccaccaccaccttccagctcagctcgggctcgcccatccacctcccggacctttcccgaatggaaccgagcctgggggcctggaagagcccaaccgcgtccagctccctttcccgtggatgaaatccaccaaagctcacgcgtggaaaggccagtgggcaggaggtgcttacacagcggaacccgaggaaaacaagaggacccgtactgcctacacccgggcgcagctgctggagctggagaaggaattcttatttaacaaatacatctcccggccccgccgggtggagctggcagtgatgttgaacttgaccgagagacacatcaaaatctggttccaaaaccgtcgcatgaagtggaaaaaagaggaagataagaaacgtagtagcgggaccccgagtgggggcggtgggggcgaagagccggagcaagattgtgcggtgacctcgggcgaggagctgctggcagtgccaccgctgccacctcccggaggtgccgtgcccccaggcgtcccagctgcagtccgggagggcctactgccttcgggccttagcgtgtcgccacagccctccagcatcgcgccactgcgaccgcaggaaccccggtgaggacagcagtctgagggtgagcgggtctgggacccagagtgtggacgtgggagcgggcagctggataagggaacttaacctaggcgtcgcacaagaagaaaattcttgagggcacgagagccagttggatagccggagagatgctgcgagcttctggaaaaacagccctgagcttctgaaaactttgaggctgcttctgatgccaagctaatggccagatctgcctctgaggactctttcctgggaccaatttagacaacctgggctccaaactgaggacaataaaaagggtacaaacttgagcgttccaatacggaccagcaggcgag

SEQ ID NO: 2 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 3 - DNA, 1861 bp, Mus musculus
  CDS (1093)..(1737)
ggatcccaaggtgatattgaacctggccaagcaatagtttctgagtagaaaggacttgagcagggaccgtctctggtcactctgtcctctttcccaggatggagtcagtctgtgaaacatggttgcacacacatttcctgacccaacccatagtggcggagagctggatagcactttgaactaatgggcgctcctcccagctgccagccaagaagacacttgactccttgatcgctggttcatttagacaagccgtttccctctctgagccaaaagaccccatgtgtaatactcaaagaagaggccttccttatatatatataggcacccccaaacctccttcatgctaccaagaaagggtctggacacatgccaaaaagaaagaggaaaaggcaaagctctccccagcggccggacgggactcttctggctgggcgaggctctttgaggaaccgagagttgctgggactgagcccgcgacgggggaggcgtggagtgggggaacaaacagagtgctgctcccctcccccgacccctgccctttgtccggaatccagctgtgctctgcgggtgggggttgtggggggaggagcgggctcgcgtggcgcagcccctgggccccctccgctgattggcccgtggtgcaggcagcagcccggcaggcacgctcctggccgggggcagagcagataaagcgtgccaggggacacacgacttgcatgcagctcagaaatccctctgggtctcatcactgcagcagtggtcgagtacctcctcggagcttttctacgacttccagacgcaatttactccaggcgagggcgcctgcagtttagcagaacttcagagggagcagagaggctcagctatccactgctgcttgacactgaccctatccactgctgcttgtcactgactgacctgctgctctctattcttttgagtcgggagaactaggtaacaattcggaaactccaaagggtggatgaggggcgcgcggggtgtgtgtgggggatactctggtcccccgtgcagtgacctctaagtcagaggctggcacacacacaccttccattttttcccaaccgcaggatggcgcctcatcccttggatgcgctcaccatccaagtgtccccagagacacaacaaccttttcccggagcctcggaccacgaagtgctcagttccaattccaccccacctagccccactctcatacctagggactgctccgaagcagaagtgggtgactgccgagggacctcgaggaagctccgcgcccgacgcggagggcgcaacaggcccaagagcgagttggcactcagcaaacagcgaagaagccggcgcaagaaggccaatgatcgggagcgcaatcgcatgcacaacctcaactcggcgctggatgcgctgcgcggtgtcctgcccaccttcccggatgacgccaaacttacaaagatcgagaccctgcgcttcgcccacaactacatctgggcactgactcagacgctgcgcatagcggaccacagcttctatggcccggagccccctgtgccctgtggagagctggggagccccggaggtggctccaacggggactggggctctatctactccccagtctcccaagcgggtaacctgagccccacggcctcattggaggaattccctggcctgcaggtgcccagctccccatcctatctgctcccgggagcactggtgttctcagacttcttgtgaagagacctgtctggctctgggtggtgggtgctagtggaaagggaggggaccagagccgtctggagtgggaggtagtggaggctctcaagcatctcgcctcttctggctttcactacttggatcc

SEQ ID NO: 4 - protein, 214 aa, Mus musculus
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 5 - DNA, 21 bp, Artificial Sequence
  Description of Artificial SequenceAdV-pdx-1 forward primer
accatgaacagtgaggagcag

SEQ ID NO: 6 - DNA, 22 bp, Artificial Sequence
  Description of Artificial SequenceAdV-pdx-1 backward primer
tcctcttgttttcctcgggttc

SEQ ID NO: 7 - DNA, 20 bp, Artificial Sequence
  Description of Artificial SequenceAdV-ngn3 forward primer
tggcactcagcaaacagcga

SEQ ID NO: 8 - DNA, 20 bp, Artificial Sequence
  Description of Artificial SequenceAdV-ngn3 backward primer
acccagagccagacaggtct

SEQ ID NO: 9 - DNA, 20 bp, Artificial Sequence
  Description of Artificial SequenceHPRT forward primer
ctcgaagtgttggatacagg

SEQ ID NO: 10 - DNA, 20 bp, Artificial Sequence
  Description of Artificial SequenceHPRT backward primer
tggcctataggctcatagtg

SEQ ID NO: 11 - DNA, 20 bp, Artificial Sequence
  Description of Artificial Sequenceendogenous pdx-1 forward primer
tggataagggaacttaacct

SEQ ID NO: 12 - DNA, 20 bp, Artificial Sequence
  Description of Artificial Sequenceendogenous pdx-1 backward primer
ttggaacgctcaagtttgta

SEQ ID NO: 13 - DNA, 20 bp, Artificial Sequence
  Description of Artificial SequenceCK19 forward primer
aagaccatcgaggacttgcg

SEQ ID NO: 14 - DNA, 20 bp, Artificial Sequence
  Description of Artificial SequenceCK19 backward primer
ctatgtcggcacgcacgtcg

SEQ ID NO: 15 - DNA, 20 bp, Artificial Sequence
  Description of Artificial Sequencenestin forward primer
ggagagtcgcttagaggtgc

SEQ ID NO: 16 - DNA, 20 bp, Artificial Sequence
  Description of Artificial Sequencenestin backward primer
gtcaggaaagccaagagaag

(19) United States
(12) Patent Application Publication
(10) Pub. No.: US 2005/0042754 A1
(43) Pub. Date: Feb. 24, 2005

(54) Induction of the formation of insulin-producing cells via gene transfer of pancreatic beta-cell-associated transcriptional factor

(76) Inventors: Jun-ichi Miyazaki, Osaka (JP); Eiji Yamato, Osaka (JP); Fumi Tashiro, Osaka (JP); Hidenori Taniguchi, Osaka (JP)

Correspondence Address: FROMMER LAWRENCE & HAUG, 745 FIFTH AVENUE- 10TH FL., NEW YORK, NY, 10151, US

(21) Appl. No.: 10/877,706

(22) Filed: Jun. 25, 2004

Related U.S. Application Data
(63) Continuation-in-part of application No. PCT/JP02/13684, filed on Dec. 26, 2002.

(30) Foreign Application Priority Data
Dec. 28, 2001 (JP) 2001-399251

(51) Int. Cl. C12N 5/08; C12N 15/85

(52) U.S. Cl. 435/455; 435/366

(57) ABSTRACT

The present invention provides a method of inducing the formation of insulin-producing cells which comprises transferring a pancreatic β-cell associated transcriptional factor gene into the pancreas to induce the formation of insulin-producing cells. The pancreatic β-cell associated transcriptional factor gene is transferred into the pancreatic tissue stem cells by the ICBD injection without ligating the common bile ducts and thus the formation of insulin-producing cells is induced. In the present invention, pdx-1, neurogenin3, etc. are used as such pancreatic β-cell associated transcriptional factor gene and an adenoviral vector with the use of the Cre-loxP recombination system, etc. is used as a vector for transferring the pancreatic β-cell associated transcriptional factor gene into the pancreas. The method of the invention enables regeneration therapy for diabetes mellitus by inducing the formation of insulin-producing cells.

### REFERENCE TO RELATED APPLICATIONS

This application is a continuation-in-part of International Patent Application PCT/JP02/13684 filed Dec. 26, 2002 and published as WO 03/057878 on Jul. 17, 2003, which claims priority from Japanese Patent Application Number 2001-399251 filed Dec. 28, 2001. Each of these applications, and each application and patent mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

### TECHNICAL FIELD

The present invention relates to a method for inducing the formation of insulin-producing cells in the pancreas, particularly to a method for inducing the formation of insulin-producing cells wherein a pancreatic β-cell-associated transcriptional factor gene is transferred to the pancreas to induce the formation of insulin-producing cells.

### BACKGROUND

The total number of β-cells, insulin-producing cells in the pancreatic islets, is observed to be decreased in the pancreas of diabetes patients, and such decrease will lead to failure in the insulin secretion (J Am Optom Assoc 69(11), 727-32, 1998). Recently, transplantation of pancreatic islets is practiced in the transplantation medicine for treating diabetes mellitus. There remains, however, many drawbacks to be solved including an insufficient number of donors for pancreatic islets for transplantation and a need for a long-term immuno-suppression against rejections after transplantation (Ann Med 33(3), 186-92, 2001).

Undifferentiated cells present in vivo that repeat proliferation and differentiation as necessary, i.e. tissue stem cells, are increasingly attracting interest in these years. Since differentiation stages of tissue stem cells are thought to be closer than ES cells to the further differentiated cells, tissue stem cells are thought to be possibly differentiated into insulin-producing cells with relative simplicity.

Certain test results have been so far reported that suggest the presence of tissue stem cells in the pancreas. Ramiya et al. have reported that the pancreatic duct epithelial cells derived from NOD mice, which are considered to be a model animal for Type I diabetes, are isolated and cultured, then induced to differentiate into insulin-producing cells (Nat Med 6(3), 278-82, 2000). Further, Bonner-Weir et al. reported that they had cultured the human-derived pancreatic duct epithelial cells and generated the insulin-positive cell population (Proc Natl Acad Sci USA 97(14), 7999-8004, 2000). All of these reports suggest the presence of pancreatic stem cells near the pancreatic ductal epithelium. As for studies in vivo, Sharma et al. reported that cross sections of the pancreas of rats that underwent pancreatectomy showed a regeneration image and that at the same time the expression of pdx-1, a pancreatic β-cell-specific transcriptional factor gene, was enhanced not only in the pancreatic duct but in pancreatic islets as well (Diabetes 48(3), 507-13, 1999). Kritzik et al. reported that neogenesis of pancreatic islet was observed in the pancreatic duct in parallel with the increase in the pancreatic ductal epithelium expressing pdx-1 which, in its origin, should be a β-cell-specific transcriptional factor, in transgenic mice that over-express IFN-γ in a pancreatic islet specific manner (Am J Physiol 269(6 PT 1), E1089-94, 1995, J Endocrinol 163(3), 523-30, 1999). These suggest that the pancreatic tissue stem cells differentiate into endocrine cells in response to signaling to activate transcriptional factors such as pdx-1.

On the other hand, adenoviral vectors that can be easily handled and that yield high efficiency as a means of gene transfer into living organisms are widely used in laboratories (Nature 371 (6500), 802-6, 1994, Science 265 (5173), 781-4, 1994). So far, intravenous injection has been mainly adapted to administrate adenoviral vectors, however this method has drawbacks in that transfer efficiency is quite poor in organs other than the liver, and in that a negative influence cannot be denied since it is a systemic administration and thus expression of foreign proteins in the other organs, though at a low level, cannot be ignored, and so on.

The intra common bile ductal (ICBD) injection is a method to overcome the drawbacks mentioned above. Peeters et al. adapted the ICBD injection as a method of gene transfer to rat livers and have reported that it is possible to carry out a transfer which is more efficient and specific as well as being lower in immune response compared to when injected intravenously (Hum Gene Ther 7(14), 1693-9, 1996). Moreover, Raper and DeMatteo reported that they had succeeded in yielding high specificity to the pancreas by ligating the common bile ducts on administering intracholedochally in order that a virus may be injected only into the pancreas (Pancreas 12(4), 401-10, 1996).

These reports, however, are only for rats and the same procedure would have an extreme difficulty when applied to mice whose pancreatic and bile ducts are fragile. It is easy to produce gene-manipulated animals for various types of genes using mice, and if, moreover, it becomes possible to transfer foreign genes as above mentioned, then it would be useful to a great extent for various studies. The ICBD injection has many features common to techniques such as the endoscopic retrograde cholangiopancreatography (ERCP) which is a bloodless and highly safe method for humans. Therefore, provided that the method can be performed without imparting excess pressure to the pancreatic duct on injection and that a moderate influx is made to be possible by not ligating the mice bile ducts, the ICBD injection is expected to be applied clinically as an efficient method for transferring a gene to the pancreas (Dig Dis Sci 45(2), 230-6, 2000).

The subject of the present invention is to provide a method for inducing the formation of the pancreatic insulin-producing cells, particularly a method for inducing the formation of insulin-producing cells wherein a pancreatic β-cell-associated transcriptional factor gene is transferred to the pancreas to induce the formation of insulin-producing cells.

The present inventors have made a keen study to overcome the subjects described above and found the following: The formation of insulin-producing cells is induced by transferring a pancreatic β-cell associated transcriptional factor gene into the pancreas, just like, for example, a transcriptional factor gene necessary for the β-cell differentiation is transferred into the pancreatic tissue stem cells to raise forced expression and induce differentiation of β-cells into the adult pancreatic endocrine cells; and an efficient gene transfer to the pancreas is made to be possible by adapting the ICBD injection as a gene transfer procedure to the pancreas and by performing moderate influx without imparting an excessive pressure on injection by not ligating the bile ducts contrary to the case for mice where the ducts are ligated. The present invention is hereby completed.

### DISCLOSURE OF THE INVENTION

The present invention comprises forming insulin-producing cells by transferring a pancreatic β-cell associated transcriptional factor gene into the pancreatic tissue cells by the ICBD injection without ligating the common bile ducts to induce the formation of insulin-producing cells.

In the present invention, pdx-1, neurogenin3, etc. are used as a pancreatic β-cell associated transcriptional factor gene and an adenoviral vector and the like is used as a vector for transferring the pancreatic β-cell associated transcriptional factor gene into the pancreas.

The present invention enables regeneration therapy for diabetes by forming insulin-producing cells through inducing the formation of insulin-producing cells by the method of the present invention. The method of the present invention for transferring a gene to the pancreas has many features common to techniques such as the endoscopic retrograde cholangiopancreatography (ERCP) which is a bloodless and highly safe method for humans and thus it is expected to be clinically applied as an efficient gene transfer procedure to the pancreas.

The present invention comprises a method for inducing the formation of insulin-producing cells wherein a pancreatic β-cell associated transcriptional factor gene is transferred to the pancreas by the intra common bile ductal (ICBD) injection to induce the formation of insulin-producing cells (“1”), a method for inducing the formation of insulin-producing cells wherein a pancreatic β-cell associated transcriptional factor gene is a gene for the differentiation-associated transcriptional factor of pancreatic β-cells and wherein induction of the formation of insulin-producing cells is induction of the differentiation of tissue stem cells present in the pancreas (“2”), the method for inducing the formation of insulin-producing cells according to “1” or “2”, wherein the pancreatic β-cell associated transcriptional factor gene is transferred to the pancreas by the ICBD injection without ligating the common bile duct (“3”), the method for inducing the formation of insulin-producing cells according to any of “1” to “3”, wherein the pancreatic β-cell associated transcriptional factor gene is pdx-1 or neurogenin3 (“4”), the method for inducing the formation of insulin-producing cells according to any of “1” to “4”, wherein the pancreatic β-cell associated transcriptional factor gene is integrated into an adenovirus vector and transferred to the pancreas (“5”), the method for inducing the formation of insulin-producing cells according to “5”, wherein the adenovirus vector is constructed using the Cre-loxP recombination system (“6”), and the method for inducing the formation of insulin-producing cells according to “5” or “6”, wherein the EGFP (efficient green fluorescent protein) reporter gene is integrated into the adenovirus vector (“7”).

The present invention also comprises regeneration therapy for diabetes using the method for inducing the formation of insulin-producing cells according to any of “1” to “7” (“8”).

### BRIEF DESCRIPTION OF DRAWINGS

FIG. 1 shows the structure of an adenoviral vector used in the examples of the present invention.

FIG. 2 shows photomicrographs of EGFP expressions detected under a fluorescent microscope. The expressions indicate the results of studying by using 293 cells, a human embryonic kidney cell line, whether the adenoviral vectors constructed in the examples of the present invention can express a gene of the interest.

FIG. 3 is the photomicrographs under a fluorescent microscope presenting the appearances of the liver and pancreas by green fluorescence of EGFP on day 10 after the injection of adenoviral vectors which were constructed by integrating a gene of the interest in the downstream of a CAG promoter and integrating IRES-EGFP in the further downstream.

FIG. 4 is the photomicrographs displaying the results of histologically studying the effect of an enhanced expression of the transcriptional factors associated with β-cell differentiation in the pancreas in the examples of the present invention. The pancreas was isolated on 7-12 days after the injection and paraffinized sections were produced which were then subjected to hematoxylin/eosin staining and insulin-immunostaining with anti-human insulin antibodies.

FIG. 5 shows the results of statistical analysis among the groups in the examples of the present invention. For quantitatively examining the expressions by enhancing the expression of the transcriptional factors associated with β-cell differentiation in the pancreas, mice pancreas that were injected with various adenoviral vectors were collected, then the randomly selected sections were produced and subjected to hematoxylin/eosin staining and to insulin-immunostaining.

FIG. 6 shows the results of quantitative comparison and analysis studying the histological changes in pancreatic cells transferred with adenoviral vectors in the examples of the present invention. Total RNA of the pancreatic tissues on day 7 after the injection was extracted and subjected to RT-PCR, then intensity of the expression is quantitatively compared and examined by densitometry.

### DETAILED DESCRIPTION

The present invention comprises introducing a pancreatic β-cell associated transcriptional factor gene into the pancreas by the ICBD injection to induce the formation of insulin-producing cells. A pancreatic β-cell associated transcriptional factor gene is transferred to the pancreas by the ICBD injection without ligating the common bile ducts in the present invention, contrary to a method previously been reported for rats where common bile ducts were ligated. As a result of not ligating the ducts, a moderate flow is made possible without imparting an excess pressure on pancreatic ducts when practicing injection and thus a gene can be efficiently transferred to the pancreas.

As a pancreatic β-cell associated transcriptional factor gene for use in the present invention, pdx-1 and neurogenin 3 (Development 127, 3533-3542, 2000) can be used advantageously. In the present invention, an adenovirus vector is used as a vector for introducing a pancreatic β-cell associated transcriptional factor gene into the pancreas. Such vector incorporates the pancreatic β-cell associated transcriptional factor gene and the gene can be transferred to the pancreas. To construct such adenovirus vectors, a method for producing adenoviral vectors using the Cre-loxP recombination system (Hum Gene Ther 10(11), 1845-52, 1999) can be employed. A pancreatic β-cell associated transcriptional factor gene to be incorporated into an adenovirus vector can be obtained by cloning a full-length cell line for the gene by RT-PCR method. For instance, the mouse pdx-1 cDNA is obtained from total RNA of MIN6 cells, which are the mouse insulinoma-derived β-cell line, and the mouse neurogenin3 cDNA from total RNA of the C57BL/6J mouse brain, by cloning their full length by RT-PCR.

In the present invention, in order to confirm whether a pancreatic β-cell associated transcriptional factor gene is transferred to the cells, EGFP (Enhanced Green Fluorescent Protein) gene is incorporated as a reporter gene into an adenoviral vector so that the cells incorporating the gene can be distinguished according to their emission of green fluorescence. A conventionally known method (Hum Gene Ther 7(14), 1693-9, 1996) can be adequately employed as a means of the ICBD injection.

The method of the present invention actualizes regeneration therapy for diabetes by attempting to regenerate insulin-producing cells through raising induction of the formation of insulin-producing cells.

### EXAMPLES

The present invention will now be explained in more detail with reference to the examples. The technical scope of the present invention, however, should not be limited to these examples.

A. Experimental Method

A-1. Test Animals

9-week-old male C57BL/6J Jcl mice, 25-28 g in body weight, were used in all the experiments. The mouse chamber was kept at a constant temperature and the mice were maintained under the 12-hour light and dark cycles without assigning any particular limitation to the water and food intakes.

A-2. Construction of Adenoviral Vectors

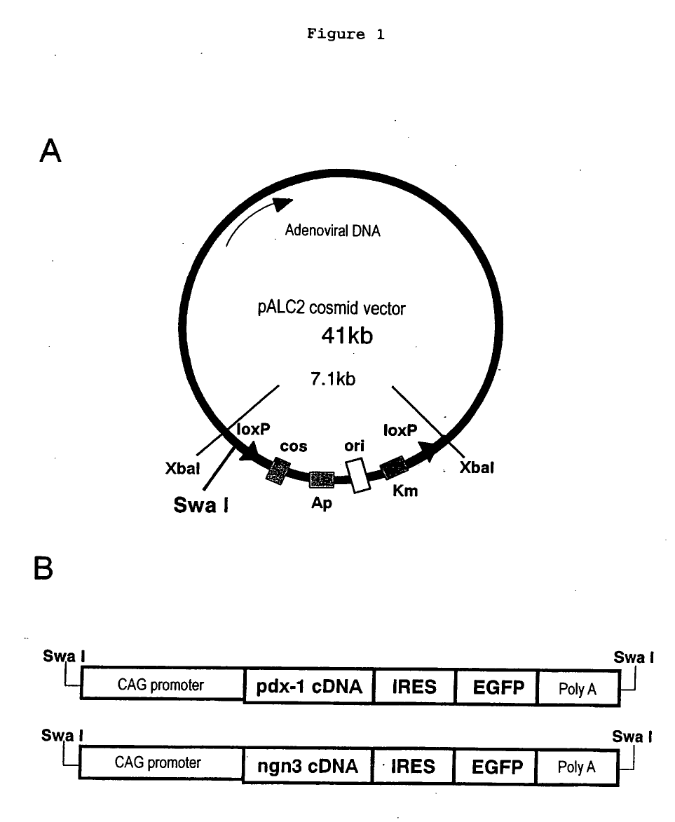

A full-length cDNA of mouse pdx-1 (SEQ. ID. No. 1) used for integrating into an adenoviral vector, was cloned from the total RNA of MIN6 cells which is a mouse insulinoma-derived β cell line, and a full-length cDNA of mouse neurogenin3 (SEQ. ID. No. 3) was cloned from the total RNA of C57BL/6J mouse brain by RT-PCR. Then their total base sequences were confirmed and used. By employing the method of producing adenoviral vectors developed in the laboratory of the present inventors which uses the Cre-loxP recombination system (Hum Gene Ther 10(11), 1845-52, 1999), three adenoviral vectors in total were produced, i.e. AdV-pdx-1, AdV-ngn3 and control free of any particular cDNA incorporation (FIG. 1). The expression cassette was incorporated with pdx-1 cDNA or neurogenin3 cDNA in the downstream of the CAG (cytomegalovirus immediate-early enhancer-chicken β-actin hybrid) promoter having a strong expression activity in many cell varieties (Gene 108 (2), 193-9, 1991), and then with IRES or EGFP in the further downstream so that the cells that received gene transfer can be distinguished by their emission of green fluorescence (FEBS Lett 470(3), 263-8, 2000). Adenoviral vectors for injection in vivo were purified by a cesium chloride gradient purification (J Virol Methods 4(6), 343-52, 1982).

A-3. Confirmation of pdx-1 Expression by Western Blotting

293 cells infected with AdV-pdx-1 were suspended in a protein extract (150 mM NaCl, 10 mM Tris-HCl (pH 7.4), 1 mM EDTA, 1% NP-40, 0.1% SDS, 0.1% sodium deoxycholate) and the cells were crushed. The protein was quantified by using Bradford assay kit (BioRad, Hercules, Calif., USA), mixed with SDS sample buffer (75 mM Tris-HCl (pH 6.8), 6% SDS, 15% glycerol, 15% 2-Mercaptoethanol, 0.015% Bromophenol Blue), and heated for 5 min at 98C°. Samples were then electrophoresed onto a 10% polyacrylamide gel and electoroblotted onto a PVDF membrane (Millipore, Bedford, Mass.). A membrane to which the protein was adsorbed was shaken for 3 hr at room temperature with the rabbit anti-mouse pdx-1 anti-serum (donated by Dr. Kajimoto, First Department of Internal Medicine, Osaka University), washed, and then shaken for a further 1 hr at room temperature with the peroxidase-bound anti-rabbit IgG antibody. Subsequently, proteins were detected by using a western blot detection kit (ECL kit; Amersham Corp., Arlington Heights, Ill., USA) based on chemiluminescense technology and by using X-ray film (FUJI MEDICAL X-RAY FILM; FUJI PHOTO FILM, Kanagawa, Japan).

A-4. Adenoviral Injection

Mice were subjected to laparotomy under general anesthesia with pentobarbital, after which a 29G needle was inserted in the retrograde fashion at the site where the common bile duct joins duodenum, and 1×10⁹ plaque forming units of adenoviral vectors adjusted with a 250 μl lactated Ringer's solution were injected as slow as to take 30 sec for the injection. Since traumatic exocrine pancreatitis may possibly be caused if an excessive pressure is imparted to the pancreatic ducts, attention was paid in order not to cause swelling. The mouse abdomens were sutured after confirming the absence of reflux. For intravenous injection, adenoviral solutions prepared similarly from the tail veins were injected (Nat Med 6(5), 568-72, 2000). Various analyses were made on 7-12 days after the experiment.

A-5. Confirming EGFP and β-Galactosidase Expressions

To confirm the EGFP expression, isolated liver and pancreas were placed on a slide glass without being fixed and subjected to a fluorescence microscopy using a 460-490-nm band-pass filter and a 510-nm long-pass filter. The isolated liver and pancreas were embedded in OTC compound (Tissue-TEC, Miles, Elkhart, Ind., USA) and were frozen with liquid nitrogen to examine the β-galactosidase expression. Then ten-βm-thick sections were produced with a frozen section producing device, fixed with 1% glutaraldehyde, and stained for 4 hr with 5-bromo-4-chloro-3-indolyl-β-D -galactosidase (X-Gal).

A-6. Histochemical Analysis

The isolated pancreas were fixed with 20% formalin solution and embedded in paraffin. Sections of 3-5 μm in thickness were produced and deparaffinized. After being dehydrated, the sections were stained with hematoxylin/eosin. Insulin-immunostaining was performed as follows. After deparaffinization, the sections were left for 10 min at room temperature with 10% normal guinea pig serum (Cosmo Bio Co. Ltd., Tokyo, Japan) to block non-specific staining. Subsequently, the sections were left for 16 hr at low temperature with the guinea pig anti-human insulin antibody (Oriental Yeast Co. Ltd., Tokyo, Japan), then with the biotinylated rabbit anti-guinea pig IgG antibody (DAKO, Kyoto, Japan) for 30 min at room temperature, and finally with 20 μl (30% hydrogen peroxidase in 100 ml PBS) of DAB (20 mg (3,3′-diaminobenzidine tetrahydrochloride) for 5 min at room temperature. These sections were washed with water before undergoing observation under a light microscope.

A-7. Evaluation of Histological Changes

Four or more random sections were prepared from the pancreas of four or more mice for each adenoviral vector and were subjected to hematoxylin/eosin staining and insulin-immunostaining for the quantitative examination of histological changes such as neogenesis of insulin-positive cells. Insulin-positive cell population was observed after allocating the cells to four groups, i.e. a group of normal pancreatic islets having five or more insulin-positive cells, a group of normal pancreatic islets having four or less insulin-positive cells, a group of newly induced pancreatic islet-like cluster with four or more cells, and a group of three or less newly induced insulin-positive cells, and comparison and analysis were made among each adenovirus-injected group.

A-8. RT-PCR

Total RNA was extracted from 293 cells on day 3 of the infection by the Acid Guanidinium-Phenol-Chloroform method to confirm the expression of adenoviral vectors in 293 cells. Reverse transcription was carried out with the total RNA as a template by using a single-stranded cDNA synthesis kit (ReverTra Ace α; TOYOBO, Tokyo, Japan). The products were amplified by PCR reaction using the Taq DNA polymerase (Gene Taq; Wako Pure Chemical Industries, Ltd., Osaka, Japan) with the use of the AdV-pdx-1 detection primer (forward primer: ACCATGAACAGTGAGGAGCAG: SEQ ID No. 5), (backward primer: TCCTCTTGTTTTCCTCGGGTTC: SEQ ID No. 6), and the AdV-ngn3 detection primer (forward primer: TGGCACTCAGCAAACAGCGA: SEQ ID No. 7), (backward primer: ACCCAGAGCCAGACAGGTCT: SEQ ID No. 8). In order to correct the differences in the total cDNA amount added to each template, PCR reaction was carried out by using HPRT detection primers (forward primer: CTCGAAGTGTTGGATACAGG: SEQ ID No. 9), (backward primer: TGGCCTATAGGCTCATAGTG: SEQ ID No. 10).

Total RNA was extracted in a similar manner as in the above from the pancreas isolated from the mice of one week after the adenoviral vector injection and was subjected to RT-PCR. In order to quantify the expressions of pdx-1 (forward primer: TGGATAAGGGAACTTAACCT: SEQ ID No. 11), (backward primer: TTGGAACGCTCAAGTTTGTA: SEQ ID No. 12), CK19 (forward primer: AAGACCATCGAGGACTTGCG: SEQ ID No. 13), (backward primer: CTATGTCGGCACGCACGTCG: SEQ ID No. 14), and nestin (forward primer: GGAGAGTCGCTTAGAGGTGC: SEQ ID No. 15), (backward primer: GTCAGGAAAGCCAAGAGAAG: SEQ ID No. 16), all of which being endogenous in the pancreas, were subjected to PCR reaction with Taq DNA polymerase (Gene Taq; Wako Pure Chemical Industries, Ltd., Osaka, Japan) using the primers for each. Electrophoresis was further performed on a 2% agarose gel, the ethidium bromide-stained gels were photographed with Fluor-Chem (Alpha Innotech, san Leandro, Calif.), and the PCR products were quantified using AlphaEase FC software (Alpha Innotech). This method was applied to quantify the mRNA expression levels as a ratio to respective HPRT expression levels for comparison and analysis.

A-9. Statistical Analysis

Student's-t test was adapted for all tests employing statistical approach and it was considered significant at p<0.05.

B. Experimental Results

B-1. Confirming the Expression in HEK 293 Cells

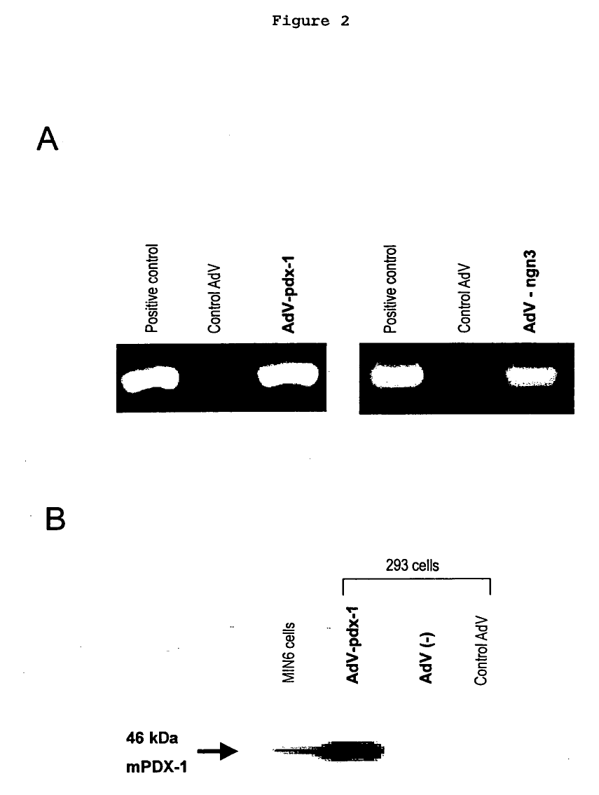

The present inventors studied whether the adenoviral vectors produced have ability to express genes of the interest with the use of 293 cells, a human embryonic kidney cell line. 293 cells were infected with AdV-pdx-1-, AdV-ngn3-, and control-adenoviral vectors at 10 moi and the EGFP expressions were detected by fluorescence microscopy on day 3 of the infection. Then the cells were collected and RT-PCR was performed. The results confirmed the mRNA expressions of the transgenes (FIG. 2A). Further, western blotting was performed for pdx-1 using proteins extracted from 293 cells. In 293 cells infected with AdV-pdx-1, a 46 kD band of pdx-1 was detected by using a protein extracted from MIN6 cells, a mouse β-cell line, as a control (FIG. 2B). Any similar band was not detected for control. Taking these results together, it was confirmed that a gene and protein of the interest were expressed in the cells owing to adenoviruses produced for the present invention.

B-2. Confirming EGFP Expression in the Liver/Pancreas

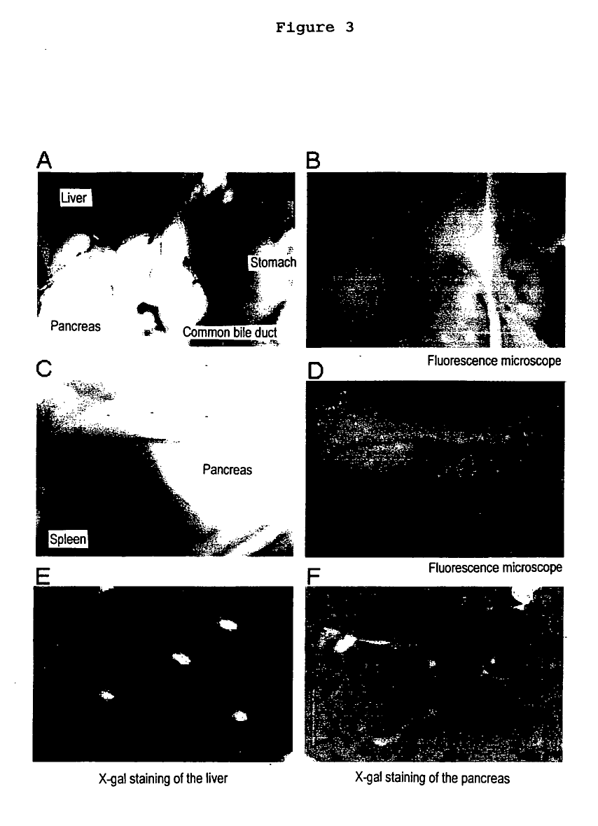

The adenoviral vectors produced in the examples of the present invention contain a gene of the interest which is integrated into the downstream of the CAG promoter and IRES-EGFP which is integrated into the further downstream, so that the gene-transferred cells are distinguishable by green fluorescence of EGFP. Ten days after the injection of adenoviruses, appearances of the liver and pancreas were observed by fluorescence microscopy (FIG. 3). Expression of the excited EGFP was obviously more intense for the ICBD injection than for intravenous injection in the liver (FIGS. 3A, B). In the pancreas, expression of EGFP was not observed at all for intravenous injection, whereas it was detected in a broad range for the ICBD injection (FIGS. 3C, D). EGFP expression was detected in neither the spleen nor stomach by both the intravenous and ICBD injections. EGFP expression was not observed for control mice administered with lactated Ringer's solution alone.

B-3. Confirmation of the Expression of β-Glactosidase Gene in the Liver/Pancreas

To further examine the efficiency of the delivery by a adenoviral vector at a tissue level, the present inventors injected AdV-LacZ, which is an adenoviral vector expressing β-glactosidase gene, in the downstream of CAG promoter (Gene, 108(2), 193-9, 1991) by the intravenous and ICBD injections, then prepared frozen sections of the liver/pancreas for X-gal staining (FIGS. 3E, F). No expression was detected at all in the pancreas by intravenous injection, whereas a broad range of expression centered on the pancreatic duct was detected by the ICBD injection (FIG. 3F). Any histologically apparent inflammation or disruption was not observed in the photomicrographs. The results above demonstrated that the ICBD injection is a considerably efficient and safe method compared to intravenous injection in transferring a gene into the pancreas by using adenoviral vectors of the present inventors. The following experiments with regard to gene transfer to the pancreas as described hereinafter employed the ICBD injection.

B-4. Histochemical Analysis of the Pancreas after the Adenoviral Vector Injection

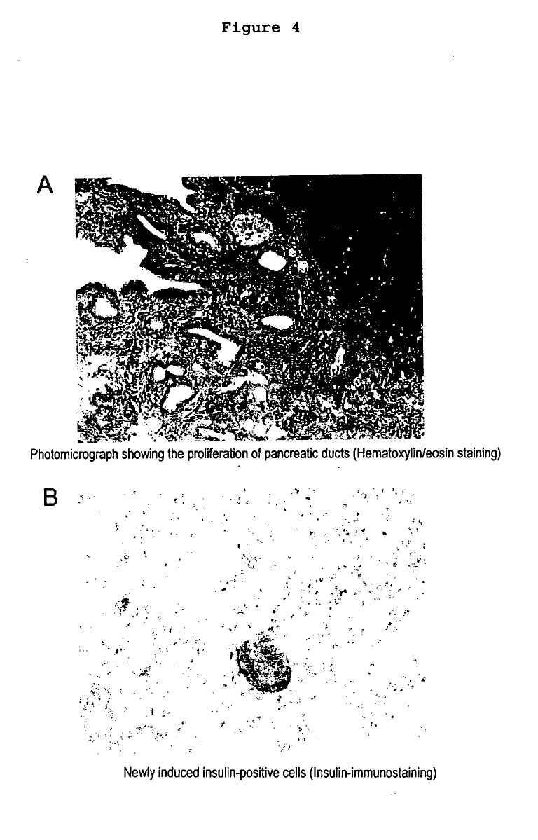

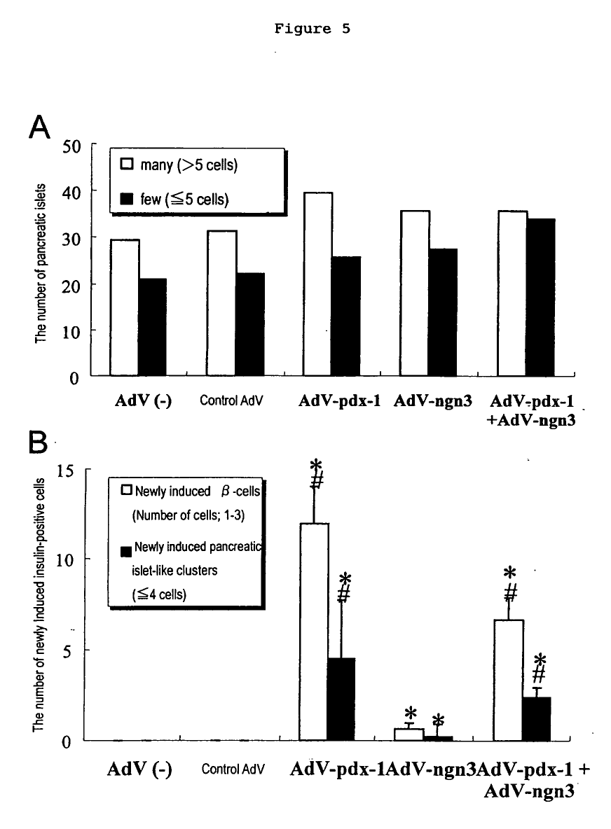

Next, the expressions of transcriptional factors associated with β-cell differentiation were enhanced in the pancreas, and the effects were histologically examined. The pancreas was isolated on 7-12 days after the injection, paraffinized sections were prepared, hematoxylin/eosin staining and insulin-immunostaining with anti-human insulin antibodies were then carried out. In the AdV-pdx-1 injected group, many regions showing the proliferation of pancreatic ducts were observed in pancreatic parenchyma in the photomicrograph of the pancreas as a result of hematoxylin/eosin staining on day 12 (FIG. 4A). In these regions, insulin-positive cells were scattered individually or as pancreatic islet-like clusters, and were considered to be neogenesis of insulin-producing cells (FIG. 4B). Such microscopic view of neogenesis was not observed in the group injected with a control adenoviral vector. In the AdV-ngn3 injected group, a similar change as that for AdV-pdx-1 was faintly observed. To study these changes quantitatively, the pancreases of mice that were injected with each adenoviral vector were collected and random sections were prepared, then stained with hematoxylin/eosin and immunostained for insulin, and statistical analysis was made among the groups. The results demonstrated that although a total number of normal pancreatic islets did not differ among the groups, the numbers of insulin-positive cells and the clusters of the cells thought to be newly generated were significantly high in the AdV-pdx-1 injected group (FIG. 5) (p<0.05). These results demonstrated that pancreatic cells to which a gene is transferred by an adenoviral vector injection to the pancreatic duct raised the proliferation of pancreatic ducts, and some of these cells were thought as having been differentiated into insulin-producing cells.

B-5. Studying Various Markers in the Pancreas by RT-PCR after the Adenoviral Vector Injection

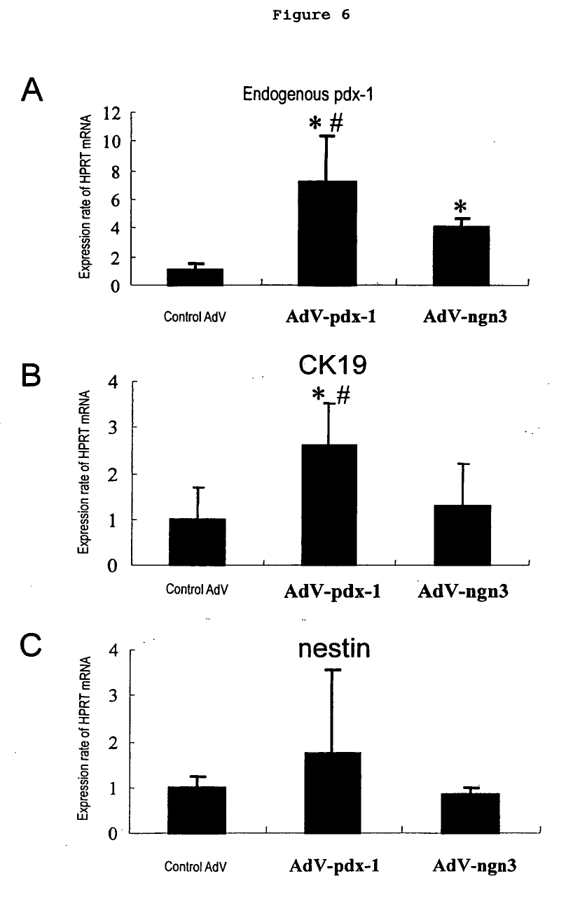

To study the cause of histological changes as shown above, total RNA of the pancreatic tissue was extracted 7 days after the injection and subjected to RT-PCR, and the intensity of the expression was compared and examined quantitatively by densitometry. As a result, activation of endogenous pdx-1 was observed in the AdV-pdx-1 injected group on day 7 of the injection (p<0.05) (FIG. 6A). The pdx-1 genes have so far been reported to possess the auto-regulatory ability due to the pdx-1 protein itself (Proc Natl Acad Sci USA, 98(3), 1065-70, 2001, Mol Cell Biol 20(20), 7583-90, 2000). This suggested that pdx-1 gene was activated by the adenoviral vector-derived pdx-1. Further, expression of CK19, a marker for the pancreatic duct, showed 2.6 fold increase with respect to controls in the AdV-pdx-1 injected group, which fact provides an evidence for the proliferation of pancreatic duct (FIG. 6B). Any data reflecting a minor change in the photomicrograph of the tissue was not obtained for AdV-ngn3. Also, expression of nestin, which was recently reported as a marker for the pancreatic endocrine progenitor cells (Diabetes 50(3), 521-33, 2001), tended to be high in the pdx-1 injected group, but there was no significant difference (FIG. 6C). This, however, only reflects the results obtained by observing mRNA expression in the whole pancreas, so that a further study will become necessary such as studying ectopic expression of nestin in the regions where the pancreatic ducts are proliferated. From the results noted above, it is considered that the endogenous pdx-1 expression was induced in the pancreas into which a transcriptional factor gene is transferred by an adenoviral vector, that the pancreatic ducts were proliferated either directly or indirectly, and that the endocrine progenitor cells were induced to differentiate into endocrine cells.

C. Discussion

The present invention focused on regeneration of the pancreatic endocrine cells by efficiently transferring a transcriptional factor gene into the pancreas and thus the experiments were carried out.

Adenoviral vectors have so far been used in many studies as a feasible and highly efficient means of gene transfer in vivo. Yet the transfer efficiency of adenoviral vectors are considerably low for the organs other than the liver when injected intravenously, nevertheless bad effects to the other organs cannot be neglected since it is a systemic administration. McClane et al. proposed a method for a specific gene transfer to the pancreas in which a needle is directly inserted into the mouse pancreatic parenchyma and although this is an ectopic administration, a certain degree of invasion would be inevitable (Hum Gene Ther 8(18), 2207-16, 1997, Pancreas 15(3), 236-45, 1997, Hum Gene Ther 8(6), 739-46, 1997). Taking these points into consideration, the present inventors employed a method to inject adenoviral vectors in a retrograde manner from the mouse common bile ducts, i.e. the intra common bile ductal (ICBD) injection method, as a specific gene transfer procedure to the pancreas. Peeters et al. adapted the ICBD injection as a method of gene transfer to rat livers and reported that a transfer which is highly efficient and specific as well as low in immune-response compared to those for intravenous injection, is possible (Hum Gene Ther 7(14), 1693-9, 1996).

On the other hand, Raper and DeMatteo reported that they had succeeded in yielding the pancreas high specificity by ligating the common bile ducts to inject viruses only to the pancreatic ducts on performing such gene transfer (Pancreas 12(4), 401-10, 1996). These are, however, reports for rats only and a similar procedure would be extremely difficult to be applied to mice in view of their fragility of the pancreatic and liver ducts. Various genetically modified animals are feasibly produced from mice hence if such foreign genes can be transferred to mice, it would be significant in deed for advancing various studies. The present inventors, therefore, did not ligate the liver ducts and accepted the loss of virus amount by the flow of virus into the liver. As a consequence, the present inventors believe it was enabled to moderately inject viruses without imparting an excess pressure to the pancreatic ducts upon injection. Confirmation of viral infection and expression in the pancreas were enabled by fluorescence microscopy because an expression cassette designed to express EGFP concomitant with a transcriptional factor of the interest was used in the present invention. As a consequence, a selective and highly efficient transfer to the liver and pancreas was actualized by the use of the ICBD injection.

Next, a differentiation-associated transcriptional factor for β-cells is studied.

Pdx-1 is a pancreatic β-cell specific transcriptional factor which functions to maintain the blood sugar homeostasis through transcriptional regulation of e.g. insulin, glucokinase or GLUT2, nonetheless it is also largely involved in development of the pancreas. Pdx-1 is expressed in the foregut in near the site where the pancreatic bud develops at embryonic stage, in the later stage of development, the expression is localized to pancreatic islets and small intestines, and is become specific to pancreatic β-cells after birth. Diabetes associated with the decrease in pancreatic β-cells is caused in humans and mice having heterozygosity of mutant pdx-1. Aplasia of the pancreas is observed in the homozygous mice (Mol Cell Biol 20(20), 7583-90, 2000, Development 122(5), 1409-16, 1996, EMBO J 13(5), 1145-56, 1994).

Neurogenin3, also a differentiation-associated transcriptional factor for β-cells, is an essential factor which determines an orientation of differentiation to the endocrine cells in the system mediated by Notch signal, when the pancreatic progenitor cells are differentiated into endocrine/exocrine cells in the pancreas in the course of developmental process of the pancreas. Expression of neurogenin3 reaches its peak on E15.5 in the pancreatic endocrine progenitor cells and disappears after birth. The pancreatic islet cells are not formed in the course of developmental process in the homo-deficient mice for neurogenin3 gene, and these mice die 1-3 days after birth from diabetes. It is found that expressions of pax4, pax6, neudoD/BETA2 were not observed in pancreases of these mice (Genes Dev 15(4), 444-54, 2001, Mol Cell Biol 20(9), 3292-307, 2000, Diabetes 49(2), 163-76, 2000, Diabetes 50(5), 928-36, 2001, Nature 400(6747), 877-81, 1999, Proc Natl Acad Sci USA 97(4), 1607-11, 2000, Mol Cell Neurosci 8(4), 221-41, 1996). From the facts above, pdx-1 and neurogenin3 contain numbers of transcriptional factors in their downstream, and the final differentiation oriented to endocrine cells in the pancreatic stem cells is thought to be controlled by pdx-1, neurogenin3, and by many transcriptional factors controlled by these two.

Recently, many attempts are made to transfer those transcriptional factors by exogenously transferring a gene and turn non-β-cells into β-cell-like cells. Watada et al. reported that when pdx-1 gene was transferred into αTC1 cells, an α-cell derived cell line, the expression of β-cell-specific proteins such as insulin and glucokinase were induced in the presence of β-cellulin (Diabetes 45(12), 1826-31, 1996). Also recently, Ferber et al. reported that when pdx-1 gene is highly expressed in the liver by intravenously administering an adenovirus which expresses the gene, some of the cells turn into insulin-producing cells, and that this method was successful in improving the blood sugar level of mice suffering STZ-induced diabetes (Nat Med 6(5), 568-72, 2000). In many cells, differentiation or trans-differentiation is not induced by introduction of a single gene and these conclusions are reached because such as an a-cell line or liver cells were used that are developmentally close to β-cells. Tissue stem cells present in the pancreas are also thought to be one of the cell populations oriented, to a certain degree, to the pancreatic endocrine/exocrine cells. The present inventors, therefore, thought that a new potentiality of the pancreatic endocrine/exocrine cells for regeneration therapy lies ahead, if pdx-1 and neurogenin3, transcriptional factors necessary for β-cell differentiation, can induce differentiation by transferring a gene to the pancreatic tissue stem cells.

Till today, although many reports have suggested that pancreatic stem cells possibly localize near the pancreatic ductal epithelium, in the pancreatic islets and so on, there localization remains largely unknown (Nat Med 6(3), 278-82, 2000, J Virol Methods 4(6), 343-52, 1982). For this reason, gene transfer was attempted using a tissue of the pancreatic parenchyma in or near the pancreatic ductal epithelium as a target in the examples of the present invention. As a result, a photomicrograph demonstrated the proliferation of pancreatic ductal epithelium itself, and the newly induced insulin-positive cells were detected in the same region. RT-PCR conducted for examining the cause for these changes revealed increase in the endogenous pdx-1 expression in the pancreas in the AdV-pdx-1 injected group. There is a binding region for the pdx-1 protein itself in the promoter region of the pdx-1 gene, where auto-regulation is taking place: This is thought to be involved in the specificity of β-cells (Proc Natl Acad Sci USA 98(3), 1065-70, 2001, Mol Cell Biol 20(20), 7583-90, 2000). What is predicted from these is that activation of endogenous pdx-1 as observed in the present invention is caused by foreign pdx-1 by an adenoviral vector.

Further, CK19, a pancreatic duct marker, was expressed significantly highly in the AdV-pdx-1 injected group and this was thought to reflect the fact that the proliferation of the pancreatic ductal epithelium was actively induced as observed in the photomicrograph of the tissue.

The present inventors thus present a presumption of the mechanism for the histological changes as follows: It is thought that many proteins are expressed in the pancreatic ductal epithelial cells as are in other cells in vivo and maintain homeostasis on the balance of activation and suppression. Although pdx-1 is only expressed at the developmental stage in the pancreatic ductal epithelial cells, pancreatic ducts may possibly be proliferated because various genes including endogenous pdx-1 are activated by enhancing the expression of pdx-1 by the foreign transfer of AdV-pdx-1 and the immature feature of the developmental stage is temporarily recalled. In the pancreatectomy models of Sharma et al. as well, it is considered that the microscopic view of dedifferentiation and redifferentiation observed in the pancreatic ductal epithelium can be explained as a result of imbalance of transcriptional factors which accompanies activation of the endogenous pdx-1 (Diabetes 48(3), 507-13, 1999), however, it is no more than a presumption. As for origin of insulin-positive cells, a possible scenario is that the tissue stem cells near the pancreatic duct which was transferred a gene by AdV-pdx-1 are affected by the expression of pdx-1 and by the transcriptional factors activated by such pdx-1 expression and that those tissue stem cells underwent final differentiation into insulin-producing cells.

As a consequence of AdV-ngn3 injection, the endogenous pdx-1 activation and tissue alterations are faintly observed, meanwhile neurogenen3 is once expressed intensively during developmental process but then fade out and the adult pancreatic islet cells appear. Therefore, the inventors studied the possibility of insulin-positive cells to appear after the transient and high expression of neurogenin3 induced by an adenoviral vector was vanished, by using the pancreas of one month after the AdV-ngn3 injection. But there was no obvious change. These results suggest that an effect as same degree as that of pdx-1 cannot be obtained by transferring neurogenen3 alone. In the present invention, the present inventors have succeeded in performing an efficient and safe gene-transfer of adenoviral vectors to the mouse pancreas with the use of the ICBD injection. When pdx-1 gene is transferred, the photomicrograph displays the proliferation of pancreatic duct in the pancreatic parenchyma, and newly induced insulin-positive cells were observed to emerge in the region. RT-PCR analysis suggested that these changes are associated with the activation of endogenous pdx-1 or CK19. Such an attempt to induce differentiation by transcriptional factors of pancreatic tissue stem cells in vivo has never been challenged and it provides a new approach for curing diabetes mellitus by gene-transfer.

### INDUSTRIAL APPLICABILITY

The present invention enables regeneration therapy for diabetes mellitus since when a pancreatic β-cell associated transcriptional factor pdx-1 gene is integrated into an adenoviral vector and administered to the mouse common bile duct, the gene is efficiently transferred to the pancreatic tissue cells to induce the formation of insulin-producing cells, which leads to regeneration of insulin-producing cells. The method of the present invention especially provides a strategy for a drastic cure for Type I diabetes.

The method of the present invention makes it possible to cause moderate flow without imparting an excessive pressure to pancreatic ducts on injection by performing the ICBD injection and without ligating bile ducts. For humans, therefore, this method has many features common to techniques such as the endoscopic retrograde cholangiopancreatography (ERCP) which is a bloodless and highly safe procedure so that the method of the present invention is expected to be clinically applied as an efficient gene transfer procedure to the pancreas. Further, the method of the present invention can be applied to mice whose pancreas and bile ducts are fragile. It is highly significant if the present method is applied to mice as experimental animals for carrying out various studies, since mice are advantageous for producing many kinds of genetically manipulated animals.

Moreover, the method of the present invention enabled a highly efficient and safe gene transfer to the pancreas by adapting adenoviral vectors for the ICBD injection and thus succeeded in inducing the formation of insulin-producing cells by transferring the gene. Induction of the formation by a transcriptional factor of the pancreatic tissue stem cells in vivo has never been attempted and the method of the present invention provides a promising approach for treating diseases such as diabetes mellitus by gene transfer.

The invention will now be further described by the following numbered paragraphs:

1. A method for inducing the formation of insulin-producing cells wherein a pancreatic β-cell associated transcriptional factor gene is transferred to the pancreas by the intra common bile ductal (ICBD) injection to induce the formation of insulin-producing cells.

2. A method for inducing the formation of insulin-producing cells wherein a pancreatic β-cell associated transcriptional factor gene is a gene for the differentiation-associated transcriptional factor of pancreatic β-cells and wherein induction of the formation of insulin-producing cells is induction of the differentiation of tissue stem cells present in the pancreas.

3. The method for inducing the formation of insulin-producing cells according to paragraph 1 or 2, wherein the pancreatic β-cell associated transcriptional factor gene is transferred to the pancreas by the ICBD injection without ligating the common bile duct.

4. The method for inducing the formation of insulin-producing cells according to any of paragraphs 1 to 3, wherein the pancreatic β-cell associated transcriptional factor gene is pdx-1 or neurogenin3.

5. The method for inducing the formation of insulin-producing cells according to any of paragraphs 1 to 4, wherein the pancreatic β-cell associated transcriptional factor gene is integrated into an adenovirus vector and transferred to the pancreas.

6. The method for inducing the formation of insulin-producing cells according to paragraph 5, wherein the adenovirus vector is constructed using the Cre-loxP recombination system.

7. The method for inducing the formation of insulin-producing cells according to paragraph 5 or 6, wherein the EGFP (efficient green fluorescent protein) reporter gene is integrated into the adenovirus vector.

8. Regeneration therapy for diabetes using the method for inducing the formation of insulin-producing cells according to any of paragraphs 1 to 7.

1. A method for inducing the formation of insulin-producing cells wherein a pancreatic β-cell associated transcriptional factor gene is transferred to the pancreas by the intra common bile ductal (ICBD) injection to induce the formation of insulin-producing cells.

2. A method for inducing the formation of insulin-producing cells wherein a pancreatic β-cell associated transcriptional factor gene is a gene for the differentiation-associated transcriptional factor of pancreatic β-cells and wherein induction of the formation of insulin-producing cells is induction of the differentiation of tissue stem cells present in the pancreas.

3. The method for inducing the formation of insulin-producing cells according to claim 1, wherein the pancreatic β-cell associated transcriptional factor gene is transferred to the pancreas by the ICBD injection without ligating the common bile duct.

4. The method for inducing the formation of insulin-producing cells according to claim 2, wherein the pancreatic β-cell associated transcriptional factor gene is transferred to the pancreas by the ICBD injection without ligating the common bile duct.

5. The method for inducing the formation of insulin-producing cells according to claim 1, wherein the pancreatic β-cell associated transcriptional factor gene is pdx-1 or neurogenin3.

6. The method for inducing the formation of insulin-producing cells according to claim 2, wherein the pancreatic β-cell associated transcriptional factor gene is pdx-1 or neurogenin3.

7. The method for inducing the formation of insulin-producing cells according to claim 1, wherein the pancreatic β-cell associated transcriptional factor gene is integrated into an adenovirus vector and transferred to the pancreas.

8. The method for inducing the formation of insulin-producing cells according to claim 2, wherein the pancreatic β-cell associated transcriptional factor gene is integrated into an adenovirus vector and transferred to the pancreas.

9. The method for inducing the formation of insulin-producing cells according to claim 7, wherein the adenovirus vector is constructed using the Cre-loxP recombination system.

10. The method for inducing the formation of insulin-producing cells according to claim 8, wherein the adenovirus vector is constructed using the Cre-loxP recombination system.

11. The method for inducing the formation of insulin-producing cells according to claim 7, wherein the EGFP (efficient green fluorescent protein) reporter gene is integrated into the adenovirus vector.

12. The method for inducing the formation of insulin-producing cells according to claim 8, wherein the EGFP (efficient green fluorescent protein) reporter gene is integrated into the adenovirus vector.

13. Regeneration therapy for diabetes using the method for inducing the formation of insulin-producing cells according to claim 1.

14. Regeneration therapy for diabetes using the method for inducing the formation of insulin-producing cells according to claim 2.
